(19) United States
(12) Patent Application Publication
(10) Pub. No.: US 2026/0315819 A1
(43) Pub. Date: Oct. 15, 2026

(54) APPARATUS AND SYSTEMS FOR SYNERGISTIC AND DIFFERENTIATED ALTERNATIVE HEALTH THERAPIES AND METHODS OF USE THEREOF

(71) Applicant: Oval Bio Syndicate, Austin, TX (US)

(72) Inventors: Shahryar Michael Kiamanesh, Austin, TX (US); Lawrence Sasso, Richmond, TX (US); James Riley Cassel, III, Austin, TX (US)

(73) Assignee: Oval Bio Syndicate, Austin, TX (US)

(21) Appl. No.: 19/648,398

(22) Filed: Apr. 15, 2026

Related U.S. Application Data
(60) Provisional application No. 63/788,976, filed on Apr. 15, 2025.

(51) Int. Cl. A61N 5/06; A61M 16/10; G16H 20/30

(52) U.S. Cl. CPC A61N 5/0613; A61M 16/1005; G16H 20/30; A61N 2005/0637; A61N 2005/0658; A61N 2005/0663; A61N 2005/0664

(57) ABSTRACT

A chamber for providing synergistic and differentiated alternative health therapies includes a body and a cover pivotably attached to the body. A plurality of light arrays are disposed within the body and the cover. A tub is disposed within the body, wherein light is able to travel through the tub. The chamber further includes a hydrogen inhalation system, an oxygen bubbler, and a heating element.

### CROSS REFERENCE TO RELATED APPLICATIONS

The present application claims the benefit of U.S. Patent Application No. 63/788,976 for APPARATUS AND SYSTEMS FOR SYNERGISTIC AND DIFFERENTIATED ALTERNATIVE HEALTH THERAPIES AND METHODS OF USE THEREOF, filed Apr. 15, 2025, the entire contents of which are incorporated herein by reference.

### FIELD OF THE PRESENT DISCLOSURE

The present disclosure is directed to apparatus and systems for synergistic and differentiated alternative health therapies and methods of use thereof. More specifically, the present disclosure is directed to apparatus and systems for synergistic and differentiated alternative health therapies directed towards enhanced Adenosine Triphosphate production.

### INTRODUCTION

In recent years, alternative health therapies have surged in popularity, with a growing body of research supporting their benefits. Among these innovative treatments are hydrogen gas therapy, oxygen nanobubble immersion, and near-infrared and red-light therapy. Each of the aforementioned therapies offer unique mechanisms for promoting personal health and well-being and enhanced mitochondrial function to improve ATP production.

Hydrogen gas therapy leverages the unique properties of molecular hydrogen (H₂) to act as a selective antioxidant. Unlike conventional antioxidants, which can neutralize both harmful and beneficial free radicals, hydrogen selectively targets the most damaging free radicals (e.g., hydroxyl radicals, peroxynitrite radicals, etc.) while also preserving other free radicals, such as superoxide, which confers a variety of beneficial effects. Hydrogen is unique in that it is the only know antioxidant to neutralize hydroxyl radicals. Thus, hydrogen's selectivity renders hydrogen gas therapy highly effective in reducing oxidative stress. Reductions in the body's exposure to oxidative stress, corresponds with a myriad of benefits including, inflammation reduction, which can help alleviate conditions such as arthritis, asthma etc. and also improve skin health by mitigating the effects of aging.

Furthermore, oxygen-based therapies, such as oxygen nanobubble therapy, are a novel approach for increasing cellular oxygen delivery. Oxygen nanobubbles are able to pass through tissue and cell membranes due to their microscopic size, thus providing an additional cellular oxygen delivery system to complement the pulmonary system. The increase in cellular oxygen can enhance metabolic processes within cells, which corresponds with improvements to energy production, healing, and overall cellular health. For example, utilization of oxygen nanobubbles in athletes has been shown to improve recovery times by increasing oxygen availability to muscle tissue.

Additionally, near-infrared therapy (NIR) and red-light therapy (RLT) are non-invasive treatments that employ low-level wavelengths of light (i.e., 600-1000 nm) to stimulate cellular activity. Unlike ultraviolet (UV) light, which can damage the skin, near-infrared and red light penetrate the skin thus enhancing cellular repair and function without causing the harm associated with UV light. Additionally, unlike near-infrared and red light, UV light does not provide a benefit to mitochondria. NIR and RLT penetrate the skin at depths of about 5 millimeters, thus said light is able to stimulate mitochondria within that range. Mitochondrial stimulation, in turn, enhances the production of Adenosine Triphosphate (ATP), leading to improved cellular functions such as faster healing and regeneration.

Leveraging hydrogen gas therapy, oxygen nanobubbles, and NIR therapy/RLT work synergistically to enhance overall cellular health and regeneration, thus offering a comprehensive approach to wellness and healing.

However, despite the synergistic benefits of combining these therapies and treatments, exploitation of them remains inefficient, as no device simultaneously employs hydrogen gas therapy, oxygen nanobubbles, and NIR therapy/RLT. Typically, to receive all of the benefits conferred by these therapies and treatments, users must experience said therapies in individual sessions, which is both costly and time consuming. Moreover, all of the requisite equipment to provide all of the aforesaid individualized therapies requires a large spatial profile. Furthermore, simultaneous utilization of said therapies compounds the benefits conferred to users, thus individual sessions for each therapeutic treatment reduces their combined efficacy.

Accordingly, it would be desirable to provide apparatus and systems able to combine alternative health therapies, as a process for exploiting the synergistic benefits of concurrent usage of said therapies. It would be further desirable to provide apparatus and systems combining said therapies to reduce a requisite spatial profile to provide all of said therapies individually.

### SUMMARY

This Summary is provided to introduce a selection of concepts in a simplified form that are further described below in the Detailed Description. This Summary is not intended to identify key features or essential features, nor is it intended to limit the scope of the claims included herewith.

According to an aspect of the present disclosure, a chamber for providing synergistic and differentiated alternative health therapies may be provided. The chamber may comprise a body and a cover pivotably attached to the body. A plurality of light arrays may be disposed within the body and the cover. A tub may be disposed within the body, wherein light may be able to travel through the tub. The chamber may further comprise a hydrogen inhalation system, an oxygen bubbler, and a heating element.

According to other aspects of the present disclosure, the chamber may include one or more of the following features. The plurality of light arrays may emit near-infrared and red light. The near-infrared and red light may have a wavelength between 600 nm and 1000 nm. The wavelength may comprise 615 nm, 675 nm, 755 nm, and 825 nm. The hydrogen inhalation system may provide a hydrogen-ambient air mixture comprising at most 4% hydrogen. The chamber may further comprise one or more speakers housed within at least one of the body and the tub. The one or more speakers may be conductive transducers configured to cause the tub to vibrate.

According to another aspect of the present disclosure, a system for providing synergistic and differentiated alternative health therapies may be provided. The system may comprise a chamber including a body, a cover pivotably attached to the body, a plurality of light arrays disposed within the body and the cover, and a tub disposed within the body. The system may further comprise a hydrogen inhalation system, an oxygen bubbler, a heating element, one or more computer processors, and a memory having stored therein machine executable instructions. The machine executable instructions, when executed by the one or more processors, may cause the system to activate a fill valve to fill the tub with a quantity of water. The instructions may cause the system to activate, after a first period of time has elapsed, the oxygen bubbler to introduce a plurality of oxygen nanobubbles to the quantity of water. The instructions may cause the system to deactivate the fill valve after the tub is filled with a specific volume of the water. The instructions may cause the system to activate the heating element to maintain a temperature of the quantity of water. The instructions may cause the system to activate the plurality of light arrays to provide a user with near-infrared and red light. The instructions may cause the system to activate the hydrogen inhalation system to provide the user with a hydrogen mixture. The instructions may cause the system to activate, after a second period of time has elapsed, a drain valve to drain the tub of the quantity of water. The instructions may cause the system to deactivate, after a third period of time has elapsed, the plurality of light arrays and the hydrogen inhalation system.

According to other aspects of the present disclosure, the system may include one or more of the following features. The system may further comprise an external tower including a tower frame, a water tank supported by the tower frame and configured to store water from a municipal water source, and a transfer valve configured to control flow of water from the water tank to the tub. The external tower may further comprise a dump valve configured to drain water from the system. The system may further comprise a float valve configured to communicate with the fill valve to trigger closure of the fill valve when the quantity of water within the tub reaches the specific volume. The oxygen bubbler may maintain a level of dissolved oxygen within the quantity of water at approximately 28 ppm. The heating element may maintain the temperature of the quantity of water at approximately 92.5 degrees Fahrenheit.

According to another aspect of the present disclosure, a method for providing synergistic and differentiated alternative health therapies via a therapeutic chamber may be provided. The method may comprise filling, via a fill valve, a tub with water. The method may comprise introducing, after a first period of time has elapsed, a plurality of oxygen nanobubbles to the water via an oxygen bubbler. The method may comprise deactivating the fill valve after the tub is filled with a specific volume of the water. The method may comprise maintaining, via a heating element, a temperature of the water. The method may comprise providing, via a plurality of light arrays, near-infrared light and red light to a user. The method may comprise providing, via a hydrogen inhalation system, a hydrogen mixture to the user. The method may comprise draining, after a second period of time has elapsed, the tub of the water via a drain valve. The method may comprise deactivating, after a third period of time has elapsed, the plurality of light arrays and the hydrogen inhalation system.

According to other aspects of the present disclosure, the method may include one or more of the following features. The first period of time may be approximately four minutes. The second period of time may be approximately thirty-eight minutes after initiation of a therapeutic session. The specific volume may be between 90 and 130 gallons. The oxygen bubbler may maintain a level of dissolved oxygen within the quantity of water at approximately 28 ppm. The heating element may maintain the temperature of the quantity of water at approximately 92.5 degrees Fahrenheit. The hydrogen mixture may comprise at most 4% hydrogen.

The foregoing general description of the illustrative embodiments and the following detailed description thereof are merely exemplary aspects of the teachings of this disclosure and are not restrictive.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 illustrates an embodiment of an environment in which the present disclosure may be practiced.

FIG. 2 illustrates an embodiment of a block diagram of an electronic device.

FIG. 3 illustrates an embodiment of a therapeutic chamber.

FIG. 4 illustrates an embodiment of the therapeutic chamber.

FIG. 5 illustrates an embodiment of an interior of the therapeutic chamber.

FIG. 6 illustrates an embodiment of the therapeutic chamber.

FIG. 7 illustrates an embodiment of the therapeutic chamber.

FIG. 8 illustrates an embodiment of the therapeutic chamber.

FIG. 9 illustrates an embodiment of the external tower.

FIG. 10 illustrates an embodiment of the external tower.

FIG. 11 illustrates an embodiment of a block diagram of a therapeutic session method for synergistic and differentiated alternative health therapies.

FIG. 12 illustrates an embodiment of a block diagram of a system for synergistic and differentiated alternative health therapies.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

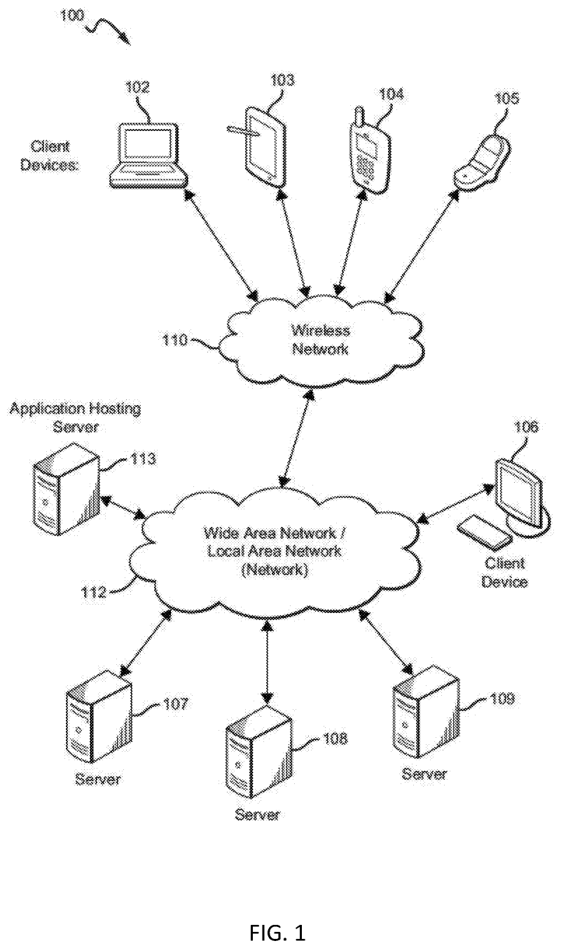

FIG. 1 illustrates components of one embodiment of an environment in which the present disclosure may be practiced. Not all of the components may be required to practice the present disclosure, and variations in the arrangement and type of the components may be made without departing from the spirit or scope of the present disclosure. As shown, the system 100 may include one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. The client devices 102-106 may include, for example, at least one of desktop computers, laptop computers, set top boxes, tablets, cell phones, smart phones, smart speakers, wearable devices (such as the Apple Watch) and the like. Servers 107-109 can include, for example, one or more application servers, content servers, search servers, and the like. FIG. 1 also illustrates application hosting server 113.

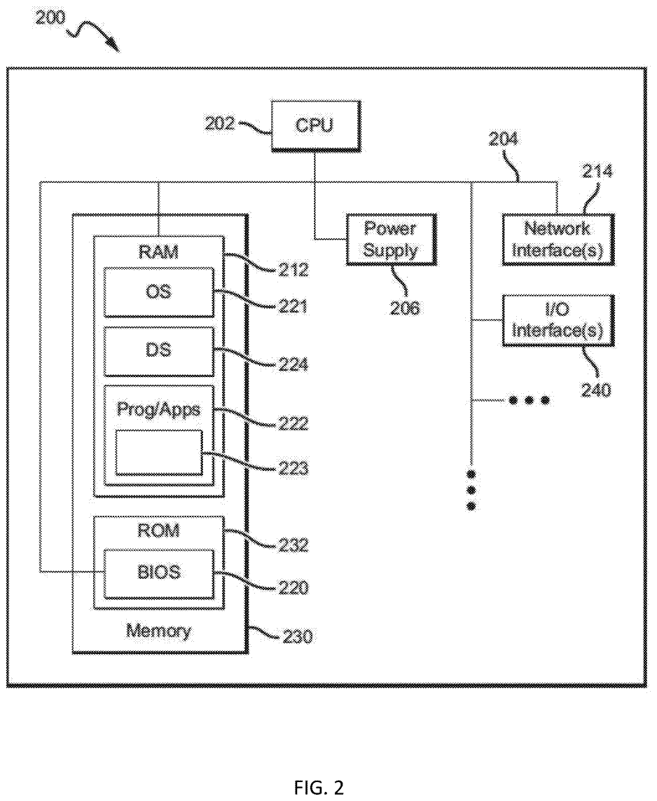

FIG. 2 illustrates a block diagram of an electronic device 200 that can implement one or more aspects of at least one of an apparatus, system and method for remote creation of dynamic advertisements (the “Engine”) according to one embodiment of the present disclosure. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the program 223. The ROM 232 can also include Basic Input/Output System (BIOS) 220 of the electronic device.

Software aspects of the program 223 are intended to broadly include or represent all programming, applications, algorithms, models, software and other tools necessary to implement or facilitate methods and systems according to embodiments of the present disclosure. The elements may exist on a single computer or be distributed among multiple computers, servers, devices or entities.

The power supply 206 contains one or more power components and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can ease processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Engine, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Engine, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games (such as fantasy sports leagues), receiving advertising, watching locally stored or streamed video, or participating in social networks.

In example aspects of the apparatus, system and method implementing the Engine, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present disclosure include apparatus, systems, and methods implementing the Engine. Embodiments of the present disclosure may be implemented on one or more of client devices 102-106, which are communicatively coupled to servers including servers 107-109. Moreover, client devices 102-106 may be communicatively (wirelessly or wired) coupled to one another. In particular, software aspects of the Engine may be implemented in the program 223. The program 223 may be implemented on one or more client devices 102-106, one or more servers 107-109, and 113, or a combination of one or more client devices 102-106, and one or more servers 107-109 and 113.

In an embodiment, the system may receive, process, generate and/or store time series data. The system may include an application programming interface (API). The API may include an API subsystem. The API subsystem may allow a data source to access data. The API subsystem may allow a third-party data source to send the data. In one example, the third-party data source may send JavaScript Object Notation (“JSON”)-encoded object data. In an embodiment, the object data may be encoded as XML-encoded object data, query parameter encoded object data, or byte-encoded object data.

Disclosed herein are apparatus, systems, and methods of use thereof for synergistic and differentiated alternative health therapies. The apparatus, systems, and methods of use thereof, may leverage a variety of alternative health therapies directed towards facilitating recovery and improving the well-being of a user. For example, the alternative health therapies may include hydrogen gas therapy, oxygen therapies (i.e., oxygen nanobubble immersion therapy), near-infrared therapy (NIR), red-light therapy (RLT), sound therapy, and/or vibrational therapy.

In an embodiment, the apparatus, systems, and methods of use thereof may employ one or more of the aforementioned alternative health therapies. A wholistic and synergistic therapy may be provided to the user, via employment of the one or more alternative health therapies. To illustrate, the apparatus, systems, and methods of use thereof may increase mitochondrial stimulation within a user's cells. Resultingly, ATP synthesis may be increased, which may correspond to increased energy levels experienced by the user. That is, increases in ATP production efficiency in the user's cells, result in more available energy for muscle contractions and brain functionality, ultimately leading to increased energy levels experienced by the user.

As a nonlimiting example, the apparatus, systems, and methods of use thereof may facilitate optimization of the electron transport chain within the user's mitochondria. Specifically, optimization of the electron transport chain may be achieved via provision of an abundance of available oxygen to the user. Increases in available oxygen may activate the user's enzymes, facilitate neutralization of free radicals within the user's body, and/or a combination of two or more of the aforementioned effects.

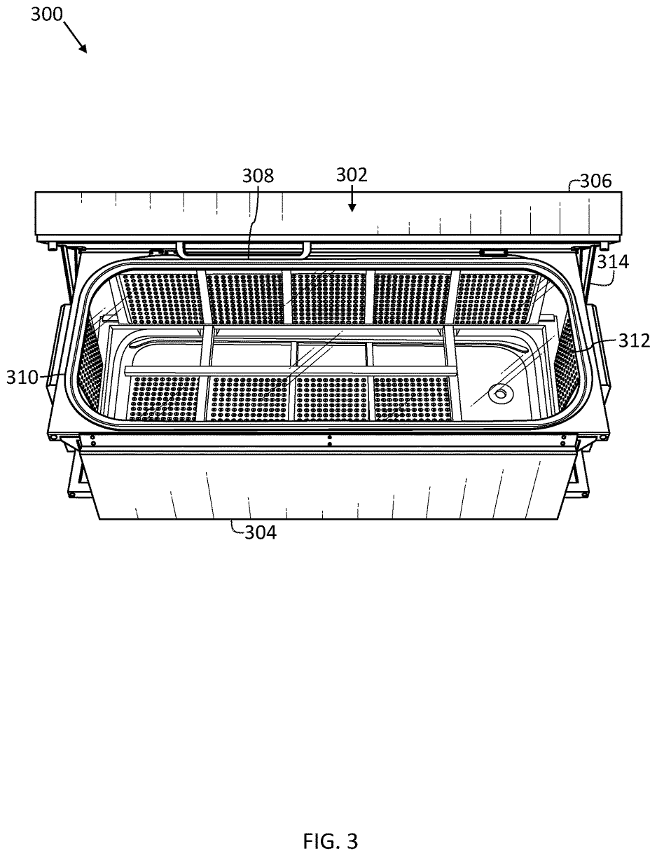

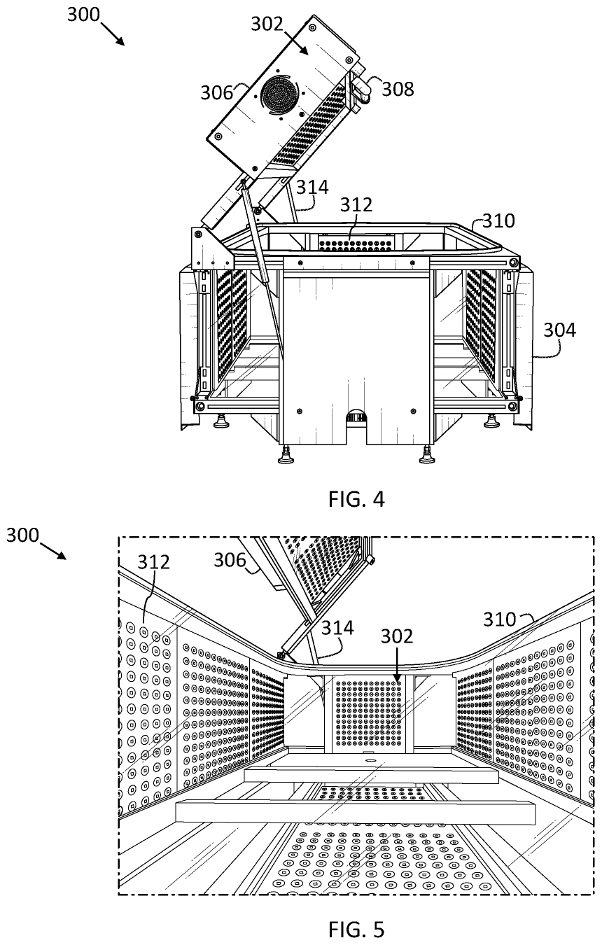

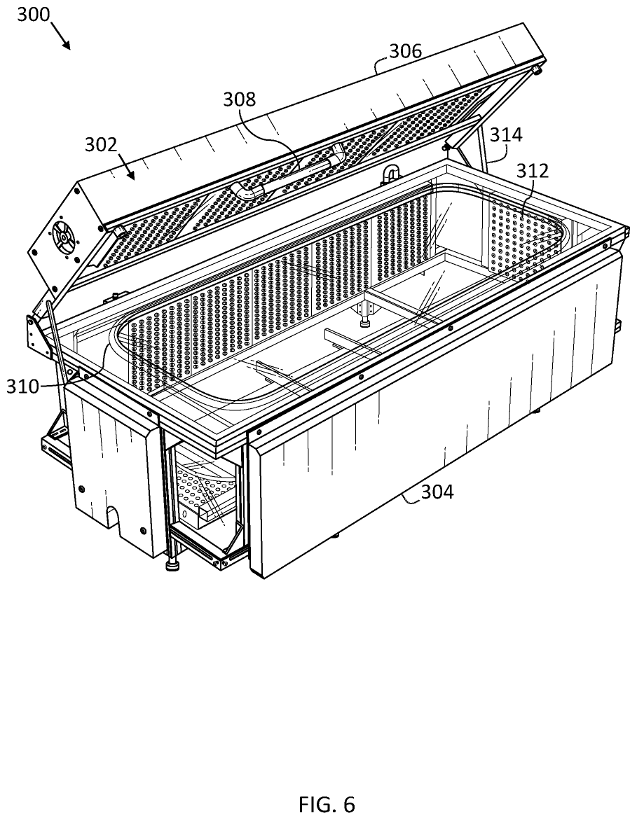

Moving to FIGS. 3-6, aspects of the present disclosure may relate to a system for synergistic and differentiated alternative health therapies (the “system”) 300. In an embodiment, the system 300 may be comprised of a therapeutic chamber (the “chamber) 302. In an embodiment, the chamber 302 may include at least one of a chamber body 304 and a chamber cover 306. For example, the cover 306 may be attached to the body 304 via one or more hinges and/or one or more dampers 314. As an illustration, the one or more hinges may pivotably attach the cover 306 to the body 304, such that the chamber 302 may be in at least one of an open position and a closed position.

Furthermore, the one or more dampers 314 may reduce the rate at which the cover 306 moves. For instance, the one or more dampers 314 may be a hydraulic door damper creating resistance when manual user actuation causes the cover 306 to move. Movement of the cover 306, via manual user actuation, may transition the chamber 302 between the open and closed positions. Additionally, the one or more dampers 314 may prevent the cover 306 from being rapidly moved via manual user actuation. That is, the one or more dampers 314 may increase a force required to close the cover 306, while simultaneously reducing the requisite force to open it 306.

Moving on, the user may enter the chamber 302 while in the open position. Upon entrance into the chamber 302, the user may situate themselves within the body 304. Meaning, the body 304 may be sized to hold an adult human. Further, once in the body 304, the user may manually actuate the cover 306 to transition the chamber 302 from the open to closed position.

In various embodiments, the body 304 may support various user positioning options, including reclined, semi-reclined, seated, or prone positions. In some aspects, the body 304 may include adjustable support surfaces or cushions to facilitate different positioning configurations.

Furthermore, the chamber 302 may include accessibility features to assist users in entering and exiting. Such features may include steps, handrails, grab bars, lift assists, or motorized platforms. In some embodiments, the body 304 may be constructed from various materials including fiberglass, acrylic, stainless steel, composite materials, or combinations thereof. The chamber 302 may also include ventilation systems or air circulation mechanisms to maintain air quality within the chamber 302 when in the closed position.

In an embodiment, the chamber cover 306 may be further comprised of a handle 308. As a nonlimiting example, the handle 308 may be disposed upon an outer edge of the chamber cover 306. In such an example, the handle 308 may be orthogonal to the cover 306.

Additionally, the chamber 302 may be further comprised of a tub 310. To illustrate, the tub 310 may be housed within an interior portion of the body 304. In an embodiment, the tub 310 may be comprised of a rigid material, such that it can hold the user and/or a quantity of water. As a nonlimiting example, the tub 310 may be transparent (described in more detail below).

In various embodiments, the tub 310 may be constructed from a variety of transparent or partially transparent materials. For example, the tub 310 may be comprised of acrylic, polycarbonate, and the like. In some aspects, the tub 310 may include optical coatings or treatments configured to enhance transmission of near-infrared and/or red light wavelengths while potentially filtering other wavelengths.

Furthermore, the tub 310 may be configured with partial transparency, wherein certain portions of the tub 310 are transparent while other portions are opaque or translucent. For instance, the bottom and sides of the tub 310 may be transparent to allow light transmission from the light arrays 312, while upper edges or rim portions may be opaque. The tub 310 may also be configured in various shapes and ergonomic configurations, including contoured shapes designed to support the user's body, reclined configurations, or seated configurations with integrated backrests.

In another embodiment, the user, when entering the chamber 302 in the open position, users may situate themselves within the tub 310. Meaning, while in the tub 310, the user may manually actuate the cover 306 to transition the chamber 302 from the open position to the closed position.

The chamber 302 may also be comprised of one or more light arrays 312 able to emit near-infrared and/or red light. For example, the near-infrared and/or red light may be of a wavelength between 600 nm and 1000 nm. Specifically, the near-infrared and/or red light may radiate at one of four wavelengths comprised of 615 nm, 675 nm, 755 nm, and 825 nm.

Furthermore, the light arrays 312 may operate in various modes including continuous wave operation, pulsed operation, or combinations thereof. In pulsed operation, the light arrays 312 may operate at various duty cycles, pulse frequencies, and pulse durations. For instance, the light arrays 312 may pulse at frequencies ranging from approximately 1 Hz to approximately 10,000 Hz, with duty cycles ranging from approximately 10% to approximately 90%.

The light arrays 312 may also include intensity or irradiance control mechanisms. In some aspects, the intensity of the near-infrared and/or red light may progressively increase during a therapeutic session, starting at a lower intensity and gradually increasing over time. The light arrays 312 may be comprised of various light source types including light-emitting diodes (LEDs), laser diodes, organic light-emitting diodes (OLEDs), or combinations thereof.

In various configurations, the light arrays 312 may be arranged as panels, strips, individual point sources, or matrices of light sources. The density of light sources within the arrays 312 may vary across different portions of the body 304 and/or cover 306 to provide targeted or uniform light distribution. In some embodiments, the light arrays 312 may be configured to emit different wavelengths in sequence or in predetermined combinations during a therapeutic session.

The one or more light arrays 312 may be housed by the body 304 and/or cover 306. To illustrate, at least one of the body 304 and the cover 306 may be recessed allowing the one or more light arrays 312 to be housed by the body 304 and/or cover 306, such that said arrays 312 are directed towards the interior portion of the body 304. In such an illustration, the tub 310, while the chamber 302 is in the open position, may interface with the one or more light arrays 312. Particularly, one or more light arrays 312 housed by the body 304 may be in direct contact with the tub 310.

As previously mentioned, the tub 310 may be transparent, thus allowing for the near-infrared and/or red light emitted from the light arrays 312 to travel through it. That is, while the user is situated within the tub 310, the near-infrared and/or red light emitted from the light arrays 312 may travel through said tub 310 and interface with the user's body. For instance, the near-infrared and/or red light, emitted by the one or more light arrays 312 housed within the body 304, may interface with a user's back and/or sides, while said user lies within the tub 310 on their back. Additionally, while the chamber 302 is in the closed position, the one or more light arrays 312, housed within both the body 304 and cover 306 may emit near-infrared and/or red light that interfaces with the entirety of the user's body (i.e., their front, back, and sides).

In an embodiment, the near-infrared and/or red light may activate one or more enzymes within the user's body. Particularly, the near-infrared and/or red light may activate cytochrome c-oxidase within the mitochondria of the user's cells. Activating the one or more enzymes may enhance electron transfer amongst the one or more enzymes and oxygen molecules. That is, the one or more enzymes may reduce oxygen, thus producing both water and hydrogen ions, which correspondingly increasing ATP production.

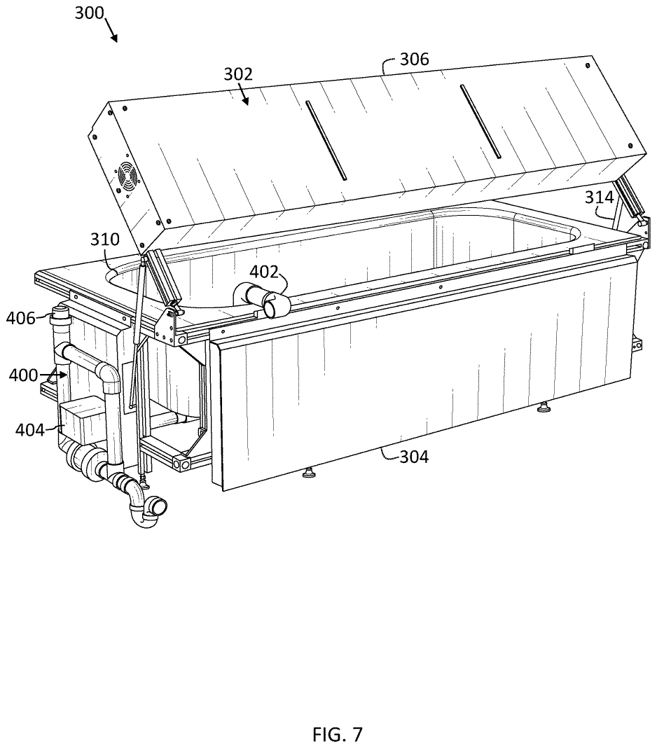

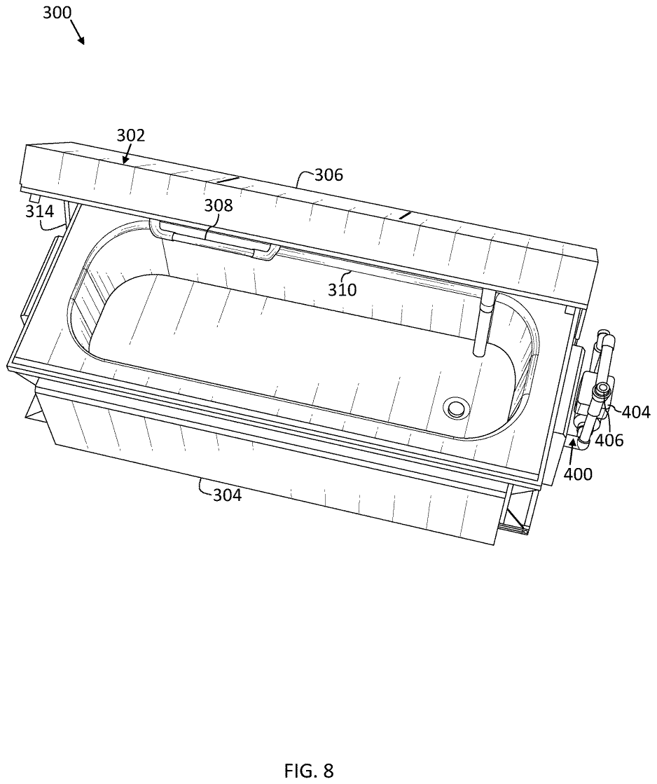

Turning to FIGS. 7 and 8, the chamber 302 may also include chamber plumbing 400. The chamber plumbing 400 may be housed within the body 304. Alternatively, the plumbing 400 may reside outside of the chamber 302. In an embodiment, the plumbing 400 may enable movement of water into and/or out of the tub 310.

To illustrate, the plumbing 400 may be comprised of one or more valves, such as a chamber fill valve 402, a chamber drain valve 404, and/or a chamber float valve 406, each serving distinct functions to ensure efficient water management.

In an embodiment, the chamber fill valve 402 may reside in at least one of an open state and a closed state. While open, water may flow from a municipal water system into a water tank 504 (described in more detail below) into the tub 310. Conversely, while closed, the chamber fill valve 402 may proscribe water from entering the tub 310.

In another embodiment, the chamber drain valve 404 may also reside in at least one of an open state and a closed state. For instance, while open, the chamber drain valve 404 may facilitate drainage of water from within the tub 310 to an outside environment. Meaning, that when in the closed state, water may remain inside the tub 310.

As a nonlimiting example, the chamber fill valve 402 and the chamber drain valve 404 may reside in opposing states. To illustrate, when the chamber fill valve 402 is open, the chamber drain valve 404 may be closed, or vice versa.

Yet further, the chamber float valve 406 may communicate to the chamber fill valve 402 when to transition between the open and closed states. For instance, water may enter the tub 310 until the chamber float valve 406 is triggered. That is, once water within the tub 310 has reached a specific volume, the chamber float valve 406 may be mechanically actuated, thus triggering the system 300 to close a tower transfer valve 508 (described in more detail below). Meaning, the chamber float valve 406 may ensure the volume of water within the tub 310 does not exceed a specific threshold. As a nonlimiting example, the specific threshold of the tub 310 may be between 90 and 130 gallons.

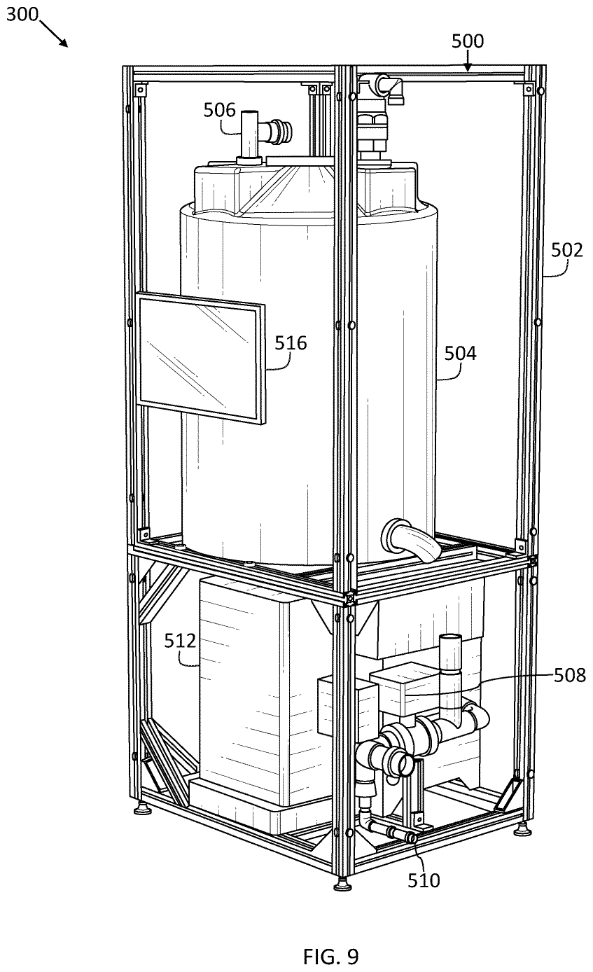

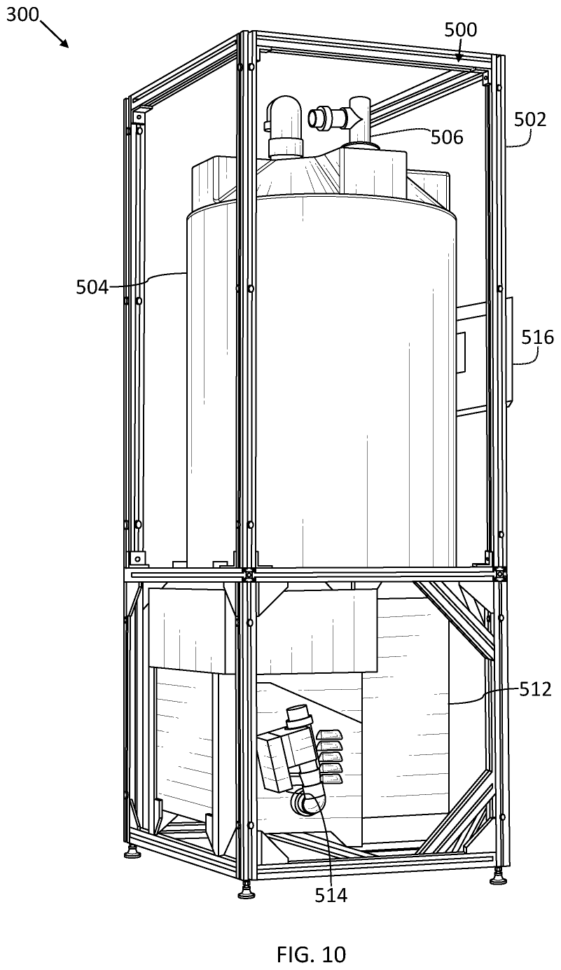

Moving on to FIGS. 9 and 10, the system 300 may be further comprised of an external tower 500. In an embodiment, the external tower 500 may be comprised of a tower frame 502 able to support an external water tank 504. For example, the water tank 504 may contain a large quantity of water, thus the tower frame 502 may be configured to support large amounts of weight.

The tower frame 502 may be engineered to withstand substantial weight, ensuring stability and safety when the water tank 504 is filled to capacity. Constructed from durable materials, the tower frame 502 may provide a robust structure capable of supporting the large volume of water necessary for the therapeutic processes within the chamber 302. The water tank 504, positioned securely within the tower frame 502, may serve as a reservoir for the water used in the chamber 302. This configuration may allow for efficient water management, facilitating the flow of water into the chamber 302 through the chamber plumbing 400.

Furthermore, the external tower 500 may be further comprised of a water tank 504. In an embodiment, the water tank 504 may be configured to store water from a municipal water source. To illustrate, the water tank 504 may be connected to the municipal water source, allowing water to flow into the tank 504 to ultimately be stored within said tank 504.

Furthermore, the external tower 500 may be further comprised of tower plumbing comprised of a fill valve 506, a transfer valve 508, a dump valve 510, and/or a temperature control valve.

As a nonlimiting example, the fill valve 506 may reside in an open or closed state. While open, water may flow from the municipal water system into the water tank 504. In such an example, the transfer valve 508 may remain in a closed state. Further, with the fill valve 506 in the closed state, the transfer valve 508 may be transitioned to the open state, such that water may flow through the transfer valve 508 into the tub 310, whereas, while closed, the transfer valve 508 may proscribe water from entering the tub 310.

In another embodiment, the water flowing through the transfer valve 508, while open, may additionally travel through the temperature control valve prior to entering the tub 310. The temperature control valve may maintain a temperature of the water entering the tub 310. For example, the water flowing into the tub 310 from the temperature control valve, may have a temperature of approximately 92.5 °F. However, the water may be maintained at any suitable temperature alternative.

In yet a further embodiment, it may take approximately four minutes for the tub 310 to fill with water, after which the user may enter the tub 310, such that they are partially immersed within the water.

Additionally, the external tower 500 may be further comprised of an oxygen bubbler 512. In an embodiment, the oxygen bubbler 512 may be enclosed within a soundproof encasing. To illustrate, such an encasing may be configured to reduce and/or completely eliminate noise generated from operation of the oxygen bubbler 512.

The oxygen bubbler 512 may introduce a plurality of oxygen nanobubbles into the water within the tub 310. As an illustration, the oxygen nanobubbles may increase a level of dissolved oxygen within the water. Specifically, the oxygen bubbler 512 may maintain the level of dissolved oxygen at approximately 28 ppm. Introduction of the oxygen nanobubbles within the water may facilitate diffusion of the dissolved oxygen from the water into a user within the tub 310. As a nonlimiting example, the dissolved oxygen may passively diffuse through the user's skin into said user's cellular membranes. Resultingly, the oxygen concentration within said user's cells may increase.

The oxygen bubbler 512 may generate nanobubbles using various methods including pressurized dissolution, ultrasonic generation, membrane diffusion, electrolysis, or combinations thereof. In some aspects, the oxygen bubbler 512 may maintain dissolved oxygen concentrations ranging from approximately 10 ppm to approximately 50 ppm, or higher. The specific concentration may be adjusted based on therapeutic requirements or user preferences.

Increasing the oxygen concentration within the user's cells may improve a user's health. In particular, the health of both the user's skin and hair may improve as the increase in cellular oxygen concentration creates an oxygen rich environment unsuitable to anaerobic bacteria (i.e., Propionibacterium). One having ordinary skill in the art will appreciate that anaerobic bacteria responsible for various skin conditions (e.g., acne vulgaris) are unable to thrive in oxygen rich environments. Thus, increasing the cellular oxygen concentration within the user's cells, via the introduction of oxygen nanobubbles within the water, enhances treatment and mitigation of skin conditions resulting from anaerobic bacteria.

Additionally, the system 300 may include a heating element 514 configured to raise and/or maintain the temperature of the quantity of water. In an embodiment, the heating element 514 may be housed within the external tower 500. However, in another embodiment, the heating element 514 may reside outside of the chamber 302.

The heating element 514 may employ various heating methods including resistive heating elements, heat pump systems, inline heaters, radiant heating, or combinations thereof. In some aspects, the system 300 may include multiple heating elements configured for zone-based heating, wherein different portions of the water within the tub 310 may be maintained at different temperatures.

Furthermore, the heating element 514 may be configured to implement temperature ramping profiles during a therapeutic session. For example, the water temperature may be gradually increased or decreased over the course of the session according to a predetermined profile. The system 300 may also include insulation around the tub 310 or within the body 304 to improve thermal efficiency and maintain consistent water temperatures.

Such an element 514 may maintain the temperature of the water after it has entered the tub 310. For instance, maintaining the water temperature may increase the kinetic energy of the oxygen nanobubbles, such that said nanobubbles more readily diffuse into the user's cells. Moreover, maintaining the water temperature may cause the user's vascular system to vasodilate, thus increasing the flow of oxygen into the user's tissues, which ultimately improves the passive diffusion of oxygen into the user's cells. Meaning, the heating element 514 may work in conjunction with the oxygen bubbler 512 to increase the rate in which oxygen passively diffuses into the user's cells.

The chamber 302 may be further comprised of a hydrogen inhalation system 316 housed within the body 304. In an alternative embodiment, the hydrogen inhalation system 316 may reside outside of the chamber 302.

The hydrogen inhalation system 316 may be comprised of a nasal cannula connected to a tank. In an embodiment, the tank may be filled with a hydrogen mixture. The nasal cannula may enable the user to inhale the hydrogen mixture.

Furthermore, the hydrogen may be prediluted with ambient air such that it is not explosive. In one example, the hydrogen mixture may be comprised of 4% hydrogen and 96% ambient air. Concentrations of hydrogen under 4% may render it inert, such that it may not combust. In an exemplary embodiment, the hydrogen mixture may be comprised of 2% hydrogen and 98% ambient air.

In one or more embodiments, the hydrogen inhalation system 316 may include safety monitoring systems configured to detect hydrogen concentrations, monitor for leaks, and automatically shut off hydrogen delivery if unsafe conditions are detected.

Moreover, the hydrogen inhalation system 316 may be configured to dispense approximately 600 mL of the hydrogen mixture per minute. Thus, user inhalation of the hydrogen mixture may facilitate the neutralization of free radicals (e.g., hydroxyl) within the user's body. Those having ordinary skill in the art may appreciate that free radicals may comprise any chemical compound that increases oxidative stress within the user's body.

Oxidative stress hampers the user's mitochondrial functionality, which in turn decreases ATP production. Meaning, neutralization of free radicals, via the hydrogen inhalation system 316, reduces oxidative stress upon the user's body, leading to an increase in ATP synthesis efficiency. As a nonlimiting example, the hydrogen inhalation system 316 increases the concentration of freely available hydrogen within the user's body, facilitating increased binding to hydroxyl groups, which in turn neutralizes said hydroxyl groups. Reducing hydroxyl groups present in a user's body, reduces the oxidative stress upon the user's body, and correspondingly improves the user's mitochondrial functionality and ATP production.

In a further embodiment, the heating element 514, the oxygen bubbler 512, the light array 312, and the hydrogen inhalation system 316 may work synergistically to increase ATP production. As a nonlimiting example, the heating element 514, the oxygen bubbler 512, and the light array 312 may work in concert to increase ATP production.

As described above, the heating element 514 and the oxygen bubbler 512 work together to increase cellular oxygen concentration, providing more available oxygen to interact with cytochrome c-oxidase activated by the near-infrared and/or red light emitted from the light array 312. Resultingly, the rate at which ATP is synthesized is increased. Additionally, neutralizing free radicals, via the hydrogen inhalation system 316, reduces oxidative stress within the user's body, which correspondingly improves both the user's mitochondrial functionality and ATP production. Meaning, the heating element 514, the oxygen bubbler 512, the light array 312, and the hydrogen inhalation system 316 work in concert to facilitate improved ATP production within the user's mitochondria.

The chamber 302 may include one or more speakers 318 (obscured). The one or more speakers 318 may be housed within the body 304 and/or the tub 310. In an embodiment, the one or more speakers 318 may be waterproof, such that said speakers 318 may interface with the water in the tub 310.

As a nonlimiting example, the one or more speakers 318 may be conductive transducers. In such an example, the conductive transducers, when applied to a surface may cause said surface to vibrate, effectively turning it into a speaker. Specifically, the conductive transducers may be applied to a surface of the tub 310, such that said tub 310, and correspondingly the quantity of water vibrate, facilitating the transmission of a variety of sounds.

In various embodiments, the one or more speakers 318 may comprise different transducer types including piezoelectric transducers, electromagnetic transducers, bone conduction transducers, or combinations thereof.

The one or more speakers 318 may play a variety of sounds. For example, one or more client devices 102-106 may connect to the speakers 318 via Bluetooth. However, any suitable connection means may enable the one or more user devices to connect to one or more speakers 318.

The variety of sounds may encompass at least one of songs, nature sounds (e.g., rainfall, ocean waves, etc.), white noise, pink noise, brown noise, binaural beats, sound bowls, and autonomous sensory meridian response sounds. In an embodiment, the variety of sounds may be configured to play at a specific frequency, which induces relaxation and/or healing within the user. For instance, the variety of sounds may play at a frequency between 432 Hz and 528 Hz. In another embodiment, the variety of sounds may be configured to promote cellular healing and/or DNA repair within the user's body.

Moreover, the variety of sounds may, in addition to inducing relaxation and/or healing within the user via the specific frequency, introduce vibrations into the quantity of water. The vibrations derived from the variety of sounds may improve circulation and/or blood flow within the user's body.

Additionally, the chamber may include a chamber control system. In an embodiment, the chamber control system may include a control panel 516 comprised of one or more computer processors and/or a memory. For example, the memory may have stored therein a set of machine executable instructions. The set of machine executable instructions may be executed by the one or more processors.

In another embodiment, the chamber control system may be implemented by one or more of the client devices 102-106 and/or the control panel 516. As a nonlimiting example, a laptop computer may implement the chamber control system such that said system initiates a therapeutic session.

In another nonlimiting example, during the therapeutic session, the chamber control system may control the functionality of at least one of the chamber plumbing 400, the tower plumbing, the oxygen bubbler 512, the heating element 514, the hydrogen inhalation system 316, the light arrays 312, and the one or more speakers 318. Further, once the therapeutic session has been initiated, the chamber control system may activate the chamber plumbing 400, the tower plumbing, the oxygen bubbler 512, the heating element 514, the hydrogen inhalation system 316, the light arrays 312, and/or the one or more speakers 318 at specific times during the therapeutic session. Yet further, the user may enter the tub 310 prior to initiation of the therapeutic session.

To illustrate, upon initiation of the therapeutic session, a method 600 to carry out the chamber control system may start with filling, via the chamber fill valve 402 and/or the fill valve 506, the tub 310 with water 602.

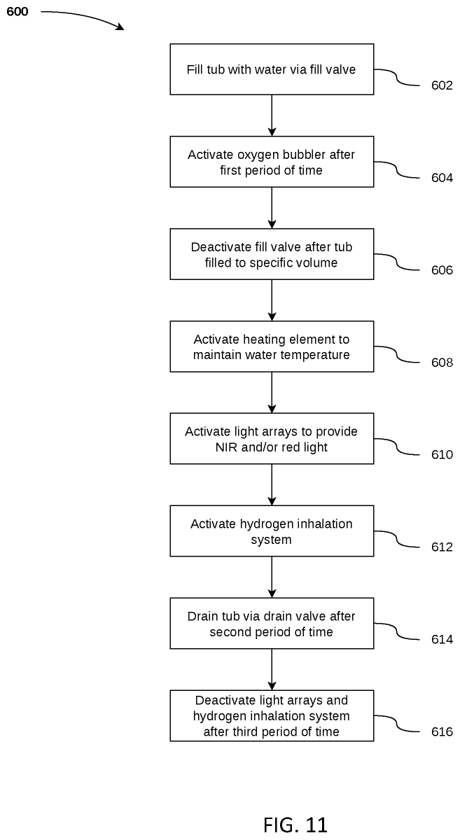

Referring to FIG. 11, aspects of the present disclosure may further relate to a therapeutic session method for synergistic and differentiated alternative health therapies (the “method”) 600. In one or more embodiments, such a method 600, may be implemented by the control panel 516. For instance, the control panel 516 may include one or more processors and memory for controlling the therapeutic session of the method 600.

In a first step 602 of the method 600, the transfer valve 508 may be activated, transitioning it from the closed state to the open state, such that water may flow from at least one of the water tank 504 and oxygen bubbler 512 to the tub 310. In such an embodiment, at the same time the transfer valve 508 is opened, the fill valve 506 may be closed.

Furthermore, in a second step 604, the control panel 516 may activate the oxygen bubbler 512 after a first period of time has elapsed to introduce oxygen nanobubbles into the water. As an illustration, the period of time may be four minutes. The activation of the oxygen bubbler 512 may be delayed by the first period of time to ensure said bubbler 512 does not run dry. To illustrate, preventing the oxygen bubbler 512 from running dry prevents it from overheating, and thus prolongs its life.

Additionally, as previously discussed, the water flowing from the transfer valve 508 into the tub 310, may first pass through the temperature control valve. The temperature control valve may ensure the water entering the tub 310 is at approximately 92.5 °F.

Water may continue to enter the tub 310 until the float valve 406 is triggered. That is, once the water within the tub 310 has reached a specific volume, the float valve 406 may be mechanically actuated, thus triggering at least one of the transfer valve 508 and the chamber fill valve 402 to close.

In a third step 606 of the method, the fill valve may be deactivated after the tub is filled to a specific volume may ensure the volume of water within the tub 310 does not exceed a specific threshold. As a nonlimiting example, the specific threshold of the tub 310 may be between 90 and 130 gallons.

Moreover, the method 600 may further comprise a fourth step 608, wherein the heating element 514 may be activated, via the control panel 516, to maintain the temperature of the water within the tub 310. For instance, if the water temperature drops below 92.5 °F, the heating element 514 may be activated until the water temperature returns to 92.5 °F. Meaning, once the water temperature has reached 92.5 °F, the heating element 514 may be deactivated.

Furthermore, the oxygen bubbler 512 may continually operate after the tub 310 has been filled with water, and the transfer valve 508 has been closed. Similar to the heating element 514, the oxygen bubbler 512 may be intermittently activated. To illustrate, the oxygen bubbler 512 may continue to introduce oxygen nanobubbles into the water until a dissolved oxygen concentration threshold has been met. More specifically, the dissolved oxygen concentration threshold of the water may be approximately 28 ppm. That is, once the dissolved oxygen concentration threshold of the water within the tub 310, the oxygen bubbler 512 may be deactivated.

Additionally, the method 600 may further comprise a fifth step 610, wherein the light arrays 312 may be activated to provide near-infrared and/or red light to a user. Upon activation of the light arrays 312, the brightness of the near-infrared and/or red light may progressively increase throughout the duration of the therapeutic session. For example, the near-infrared and/or red may start at a lower brightness when the therapeutic session has first been initiated, relative to the near-infrared and/or red-light brightness as the therapeutic session progresses. Furthermore, the wavelength of the near-infrared and/or red light emitted by the light arrays 312 may be adjusted and/or controlled by the control panel 516 during the therapeutic sessions.

Yet further, in a sixth step 612 of the method 600, the hydrogen inhalation system 316 may be activated. Prior to activation, the user may insert the nasal cannula into their nostrils. After activation, the user may begin to inhale the hydrogen-ambient air mixture as described above.

In an embodiment, the method 600 may further comprise a seventh step 614, wherein the tub 310 is drained, via a drain valve, after a second period of time has elapsed. As an illustration, the second period of time may be thirty-eight minutes after the therapeutic session has been initiated. In an embodiment, upon activation of the drain valve 404 the water exits said tub 310 via the drain valve 404.

Furthermore, the user may remain in the tub 310 after it has been drained of water. In an example, the light arrays 312 and/or the hydrogen inhalation system 316 may remain operation for a third period of time.

After the third period of time has elapsed, the method 600 may proceed to a seventh step 614 wherein the light arrays and the hydrogen inhalation system 316 are deactivated. The third period of time may be seven minutes. Meaning, the therapeutic sessions lasts for a duration of approximately forty-five minutes.

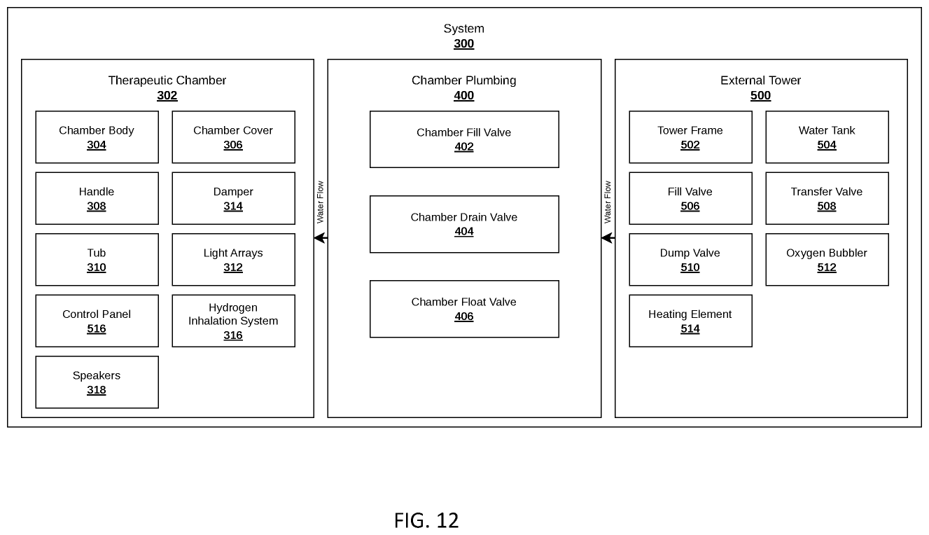

Lastly, referring to FIG. 12, an embodiment of the overall system 300 is illustrated. The system 300 includes the therapeutic chamber 302 and the external tower 500 as primary subsystems. The therapeutic chamber 302 includes the chamber body 304, the chamber cover 306, and the handle 308. The tub 310 is disposed within the chamber body 304 to hold a quantity of water and accommodate a user. The light arrays 312 are disposed within the chamber body 304 and the chamber cover 306 to emit near-infrared and red light toward the user.

The damper 314 connects the chamber cover 306 to the chamber body 304 to control the rate of movement during opening and closing. The chamber plumbing 400 includes the chamber fill valve 402, the chamber drain valve 404, and the chamber float valve 406 for managing water flow into and out of the tub 310.

The external tower 500 includes the tower frame 502 providing structural support and the water tank 504 for storing water from a municipal water source. The fill valve 506 controls water flow into the water tank 504, while the transfer valve 508 controls water flow from the water tank 504 to the tub 310. The dump valve 510 provides a means for draining water from the system 300. The oxygen bubbler 512 introduces oxygen nanobubbles into the water to increase dissolved oxygen levels, and the heating element 514 maintains the temperature of the water.

The control panel 516 includes one or more processors and memory for controlling the system 300 components during therapeutic sessions. As illustrated, the transfer valve 508, the oxygen bubbler 512, and the heating element 514 may each individually connect to the tub 310 to facilitate the synergistic therapeutic treatments described herein. Alternatively, the oxygen bubbler 512 and/or heating element 514 may connect to the transfer valve 508.

Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only.

What is claimed is:

1. A chamber for providing synergistic and differentiated alternative health therapies comprising:
a body:
a cover pivotably attached to the body;
a plurality of light arrays disposed within the body and the cover;
a tub disposed within the body,
wherein light is able to travel through the tub;
a hydrogen inhalation system;
an oxygen bubbler; and
a heating element.

2. The chamber of claim 1, wherein the plurality of light arrays emit near-infrared and red light.

3. The chamber of claim 2, wherein the near-infrared and red light have a wavelength between 600 nm and 1000 nm.

4. The chamber of claim 3, wherein the wavelength comprises 615 nm, 675 nm, 755 nm, and 825 nm.

5. The chamber of claim 1, wherein the hydrogen inhalation system provides a hydrogen-ambient air mixture comprising at most 4% hydrogen.

6. The chamber of claim 1, further comprising one or more speakers housed within at least one of the body and the tub.

7. The chamber of claim 6, wherein the one or more speakers are conductive transducers configured to cause the tub to vibrate.

8. A system for providing synergistic and differentiated alternative health therapies comprising:
a chamber including:
a body,
a cover pivotably attached to the body,
a plurality of light arrays disposed within the body and the cover, and
a tub disposed within the body;
a hydrogen inhalation system;
an oxygen bubbler; and
a heating element;
one or more computer processors; and
a memory having stored therein machine executable instructions, that when executed by the one or more processors, cause the system to:
activate a fill valve to fill the tub with a quantity of water;
activate, after a first period of time has elapsed, the oxygen bubbler to introduce a plurality of oxygen nanobubbles to the quantity of water;
deactivate the fill valve after the tub is filled with a specific volume of the water;
activate the heating element to maintain a temperature of the quantity of water;
activate the plurality of light arrays to provide a user with near-infrared and red light;
activate the hydrogen inhalation system to provide the user with a hydrogen mixture;
activate, after a second period of time has elapsed, a drain valve to drain the tub of the quantity of water; and
deactivate, after a third period of time has elapsed, the plurality of light arrays and the hydrogen inhalation system.

9. The system of claim 8, further comprising an external tower including:
a tower frame;
a water tank supported by the tower frame and configured to store water from a municipal water source; and
a transfer valve configured to control flow of water from the water tank to the tub.

10. The system of claim 9, wherein the external tower further comprises a dump valve configured to drain water from the system.

11. The system of claim 8, further comprising a float valve configured to communicate with the fill valve to trigger closure of the fill valve when the quantity of water within the tub reaches the specific volume.

12. The system of claim 8, wherein the oxygen bubbler maintains a level of dissolved oxygen within the quantity of water at approximately 28 ppm.

13. The system of claim 8, wherein the heating element maintain the temperature of the quantity of water at approximately 92.5 degrees Fahrenheit.

14. A method for providing synergistic and differentiated alternative health therapies via a therapeutic chamber, the method comprising:
filling, via a fill valve, a tub with water;
introducing, after a first period of time has elapsed, a plurality of oxygen nanobubbles to the water via an oxygen bubbler;
deactivating the fill valve after the tub is filled with a specific volume of the water;
maintaining, via a heating element, a temperature of the water;
providing, via a plurality of light arrays, near-infrared light and red light to a user;
providing, via a hydrogen inhalation system, a hydrogen mixture to the user;
draining, after a second period of time has elapsed, the tub of the water via a drain valve; and
deactivating, after a third period of time has elapsed, the plurality of light arrays and the hydrogen inhalation system.

15. The method of claim 14, wherein the first period of time is approximately four minutes.

16. The method of claim 14, wherein the second period of time is approximately thirty-eight minutes after initiation of a therapeutic session.

17. The method of claim 14, wherein the specific volume is between 90 and 130 gallons.

18. The method of claim 14, wherein the oxygen bubbler maintains a level of dissolved oxygen within the quantity of water at approximately 28 ppm.

19. The method of claim 14, wherein the heating element maintain the temperature of the quantity of water at approximately 92.5 degrees Fahrenheit.

20. The method of claim 14, wherein the hydrogen mixture comprises at most 4% hydrogen.
